SEQUENCE LISTING (7 sequences)

SEQ ID NO: 1 - DNA, 26 bp, Artificial Sequence
  Description of Artificial Sequence Synthetic primer
catatgcgaccctctgggagaaaatc

SEQ ID NO: 2 - DNA, 26 bp, Artificial Sequence
  Description of Artificial Sequence Synthetic primer
ggatcctctactcgtcctcctggaag

SEQ ID NO: 3 - DNA, 38 bp, Artificial Sequence
  Description of Artificial Sequence Synthetic primer
gaattcgaggctgaagctcgaccctctgggagaaaatc

SEQ ID NO: 4 - DNA, 26 bp, Artificial Sequence
  Description of Artificial Sequence Synthetic primer
ctcgagctactcgtcctcctggaagt

SEQ ID NO: 5 - DNA, 804 bp, Homo sapiens
gaccgggaccwctgkmtcgaacgatgagatttccttcaaytttactcrcrmkagwwttattcgcagcatccyccgcattagctgstccmgtcaacactacmacmgaagatgaaacsgcmcmaattccggctgaagctgtcmtcsgttactcmgatttagaaggggatttcsatgttgctgttttgccmttttccmacmgcmcaaataacsggttattgtttataaatacyactattgccmgcattgctgctaaagaagaaggggtatctctcgagaaaagagaggctgaagctgaattcgaggctgaagctcgaccctctgggagaaaatccagcaagatgcaagccttcagaatctgggatgttaaccagaagaccttctatctgaggaacaaccaactagttgccggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagctcgagaatcactagkgaattcmcgtggccagcc

SEQ ID NO: 6 - DNA, 459 bp, Homo sapiens
cgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtag

SEQ ID NO: 7 - DNA, 755 bp, Homo sapiens
gcattgctgctaaagaagaaggggtatctytcgagaaaagagaggctgaagctgaattcgaggctgaagctcgacctctgggagaaaatccagcaagatgcaagcttcagaatctgggatgttaaccagaagacttctatctgaggaacaaccaactagttgccggatacttgcaaggaccaaatgtcaatttagaagaaaagatagatgtggtacccattgagcctcatgctctgttcttkgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccwatatgcctgacgaaggcgtcatggtcaccaaattctacktccaggrggasgagtagctcgrgaatcactagtgaattcacgkggcccagccggcsgtctsggatcggtacctsgagccgsggsggccsccagctttytrgaacaaaaactcatctcagargrggatckgaawagsgcsgtsgaccatcatcatcatcatcatkgagttkgtasccttagacakgackgttcctcagttcawgyygkscmcktcagaagaccggtcgtgctwgatctwtcamgt

(19) United States
(12) Patent Application Publication
(10) Pub. No.: US 2008/0166762 A1
(43) Pub. Date: Jul. 10, 2008

(54) HETEROLOGOUS PRODUCTION OF INTERLEUKIN 1 RECEPTOR ANTAGONIST (IL-1Ra) IN PICHIA PASTORIS

(75) Inventors: Livy Shivraj, Navi Mumbai (IN); Pradnya Palande, Navi Mumbai (IN); Venkata Ramana Kondiboyina, Navi Mumbai (IN)

Correspondence Address: FOLEY AND LARDNER LLP;SUITE 500, 3000 K STREET NW, WASHINGTON, DC, 20007, US

(73) Assignee: Reliance Life Science PVT.LTD.

(21) Appl. No.: 11/861,914

(22) Filed: Sep. 26, 2007

(30) Foreign Application Priority Data
Sep. 27, 2006 (IN) 1565/MUM/2006

(51) Int. Cl. C12P 21/04

(52) U.S. Cl. 435/69.1

(57) ABSTRACT

The invention relates to the production and expression of Interleukin 1 receptor antagonist (IL-1Ra) in expression systems, such as Pichia Pastoris (P. pastoris). For example, the present invention encompasses an engineered Pichia strain x-33/pGAPZαA/IL-1Ra constructed by cloning the IL-1Ra gene in pGAPZαA and transforming the recombinant vector into Pichia Pastoris x-33.

### CROSS-REFERENCES TO RELATED APPLICATIONS

This application claims benefit of provisional Indian Application No., 1565/MUM/2006, filed on Sep. 27, 2006, which is hereby entirely incorporated by reference.

### FIELD OF THE INVENTION

The present invention relates to an efficient process for the production of a heterologous protein, Interleukin 1 receptor antagonist (IL-1Ra). The invention particularly relates to high yield production and expression of IL-1Ra in a Pichia Pastoris (P. pastoris) expression system.

### BACKGROUND OF THE INVENTION

### Physiological Role

Cytokines are low molecular weight, soluble proteins with a variety of functions and actions. For example, cytokines are produced in response to the presence of antigens, they function as chemical messengers for regulating the innate and adaptive immune systems, and they act as intercellular messengers for signaling a variety of cell functions. Cytokines also activate and deactivate phagocytes and immune defense cells, increase or decrease the functions of different immune defense cells, and promote or inhibit a variety of nonspecific body defenses. They are produced by virtually all cells involved in innate and adaptive immunity, but especially by T helper lymphocytes. The activation of cytokine-producing cells triggers them to synthesize and secrete their respective cytokines. The cytokines, in turn, are then able to bind to specific cytokine receptors on other cells of the immune system and influence their activity in some manner. Cytokines are pleiotropic, redundant, and multifunctional.

Cytokine receptors are broadly grouped into several families, depending on their characteristic structures. The most common type of receptor is the hemopoietin-receptor family, named because it was initially characterized by an erythropoietin receptor. The receptors of this family are heterodimers or heterotrimers and include an α and a β chain, the latter having longer intracytoplasmic segment and signaling functions. The receptors for interleukin (IL)-2, IL-3, IL-4, IL-5, IL-6, IL-7, IL-9 and IL-15 are included in this family, and some of them share subunits. Receptors for IL-3 and IL-5 share a common chain. A different β chain is shared by receptors for IL-6 and IL-11. Receptors for IL-2, IL-4, IL-7, IL-9 and IL-15 share a third chain (γ), which plays a significant role in signal transduction.

Another common type of receptor is the chemokine family. One common feature to receptors in this family is a seven transmembrane domain. Chemokines comprise a large family of structurally homologous cytokines that share the ability to stimulate leukocyte motility (chemokinesis) and directed movement (chemotaxis). The term “chemokines” is a contraction of the phrase “chemotactic cytokines.” Members of this family include IL-8, platelet factor 4, and macrophage chemotactic and activating proteins including monocyte chemo attractant protein-1 (MCP-1), MCP-2, and MCP-3. They act on neutrophil as mediators of acute inflammation.

The MCP-1 receptor has a seven transmembrane spanning receptor, which binds and murine macrophage inflammatory protein (MIP)-1. This receptor mediates mobilization of intracellular calcium in response to MCP-1, but not to related chemokines (Charo et al., Proc. Natl. Acad. Sci. (1994), Vol. 91, pp. 2752-2756).

A third family of cytokines is the Interleukin 1 superfamily, which includes two agonists, IL-1α, IL-1β, and a naturally occurring receptor antagonist, IL-1 receptor antagonist (IL-1Ra). Three linked genes, mapping within a 430-kb region of the long arm of chromosome 2 in humans, encode the secreted glycoproteins IL-1α, IL-1β and IL-1Ra. All three molecules bind to IL-1 receptors. IL-1α and IL-1β are potent proinflammatory cytokines. IL-1Ra, as an anti-inflammatory cytokine, competes with IL-1α and IL-1β in binding to IL-1 receptors without intrinsic effects. Polymorphisms exist for all three genes. The polymorphism of IL-1α, IL-1β and IL-1Ra lead to differing protein expressions.

IL-1α and IL-1β bind high affinity receptors, which are present on most nucleated cell types. The numbers of receptors range from 50 or fewer on T lymphocytes to several thousands on fibroblast cells (Oppenheim et al., Medical Immunology (2001), 10^{th} Ed., Chapter 10). Two different receptors (type I and II), both transmembrane glycoproteins, bind IL-1α and IL-1β equally. These receptors share only 28% sequence similarity. IL-1 receptor type I (IL-1-RI) is 517 amino acids long, has a 217 amino acid cytoplasmic tail and transmits signals intra-cellularly when it binds IL-1. It is responsible for signaling all IL-1 responsive cells. IL-1 receptor type II (IL-1-RII) has only 29 amino acids cytoplasmic and cannot transduce signals. The extracellular domain of IL-1RII is released in soluble form at sites of local inflammation and into the serum during systemic inflammation.

IL-1Ra has a 30% amino acid sequence homology to IL-1β and binds to human IL-1 types I and II receptors without apparent cellular activation. IL-1Ra is produced locally in various tissues in response to infection or inflammation, and is present in high levels in the circulation, secondary to hepatic production as an acute-phase protein. Multiple isoforms of IL-1Ra have been described. One isoform is a 17 kDa form, which is secreted from monocytes and other cells as glycosylated proteins of 22-25 kDa (sIL-1Ra). Other isoforms include at least three intracellular molecules (icIL-1Ra1, icIL-1Ra2, and icIL-1Ra3), which predominate in epithelial cells and fibroblasts, and are delayed products of transcription in monocytes. The icIL-1Ra isoforms may be storage forms that are released upon cell death to limit inflammation caused by cell debris (Muzio et al., J. Exp. Med. (1995), Vol. 182, Issue 2, pp. 623-628).

Functionally, IL-1Ra partially regulates the agonist effects of IL-1. IL-1Ra also blocks the in vitro stimulatory effects of IL-1 on thymocytes, fibroblasts, endothelial cells and bone cells. In addition, IL-1Ra is a potent inhibitor of the inflammatory effects of IL-1 in vivo. For example, IL-1Ra is found on T lymphocytes and endothelial cells, thereby inhibiting IL-1 activity in these cells (Cruse et al., Cytokines, Atlas of Immunology (1998), Chapter 10). IL-1Ra may be important in modulating IL-1 effects in both normal and abnormal physiology. IL-1Ra is also released by monocytes and tissue macrophages, and inhibits prostaglandin production by synovial cells and chondrocytes. In activated synovial cells, IL-1Ra inhibits Matrix metalloproteinase production (Smith et al., Arthritis and Rheum. (1991), Vol. 34, Issue 78) (Choy E. H., Exp. Op. Invest. Drugs (1998), Vol. 7, Issue 7, pp. L1087-L1097)).

Evidence suggests that IL-1 is an important mediator of inflammation and joint damage through cartilage resorption in experimental arthritis (Firestein et al., Arthritis and Rheum. (1994), Vol. 37, pp. 644-652). IL-1 induces prostaglanden (PGE2) release by synovial cells and regulates the production of numerous cytokines involved in synovitis, such as IL-1, IL-4, IL-10, IL-5 and TNE α. IL-1 also amplifies the T cell activation by inducing IL2 and IL-2 receptor gene expression, but is not required for T cell proliferation (Sany et al., Ann. Rheum Dis. (1999), Vol. 58, pp. 136-141).

In IL-1RI deficient mice, splenocytes and lymph node cells produce increased amount of IL-4 and IL-10 after antigenic stimulation (Paleolog et al., Arthritis and Rheum. (1996), Vol. 39, pp. 1082-1091). This data demonstrates that IL-1 negatively regulates IL-4 and IL-10 expression and favors the Th-1 response. Although IL-1 is a known activator of collagenase and stromelysin, an important step in cartilage breakdown, the effects of IL-1 are counterbalanced by the neutral antagonist IL-1Ra and soluble IL-1 receptors I and II (IL-1RI and IL-1RII) (Sany et al., Ann. Rheum Dis. (1999), Vol. 58, pp. 136-141).

Recombinant human soluble IL-1RI, when administrated to patients with rheumatoid arthritis, failed to produce significant clinical improvement with reduction in monocyte surface. (Drevlow et al., Arthritis Rheum. (1996), Vol. 39, pp. 257-265). In many cases, IL-1RI worsened the disease. This may be due to recombinant soluble IL-1RI binding to endogenous IL-1Ra. If this binding occurs, less IL-1Ra will bind to endogenous cell surface IL-1RI. These endogenous cell surface IL-1RI molecules will therefore engage with IL-1 itself, augmenting IL-1 induced cell activation and inflammation. As soluble IL-1RII does not bind to IL-1Ra, it may be a better therapeutic choice treating patients with rheumatoid arthritis (Choy et al., Immunotherapies, Rheumatology (1998), Chapter 10). Recombinant human IL-1Ra administered as subcutaneous injections has been tested in a double-blind placebo controlled multicenter trial (Bresnihan et al., Arthritis and Rheum. (1996), Vol. 39(supp.), pp. 73A). A dose of 150 mg/day administered subcutaneously produced significant clinical improvement (Choy et al., Rheumatology (1998), Chapter 10).

The role of cytokines in rheumatoid arthritis, and the rationale for inhibition of IL-1 and tumor necrosis factor (TNF)-α in this disease have been extensively reviewed. Much evidence indicates that both of these proinflammatory cytokines are overproduced in the rheumatoid joint and are key mediators in both inflammation and tissue destruction. The demonstrated success of the therapeutic administration of inhibitors of IL-1 and TNF-α offer further support for the importance of these cytokines in the rheumatoid disease process. However, much less is known about the importance and role of natural mechanisms to counteract the effects of IL-1 and TNF-α in the joint, and whether an imbalance in these mechanisms may predispose to the development of rheumatoid arthritis. Endogenous inhibitors of IL-1 and TNF-α include their respective soluble receptors. IL-1 effects may be further blocked by IL-1Ra.

### SUMMARY OF THE INVENTION

The present invention provides an efficient process by which Interleukin 1 receptor antagonist (IL-1Ra) can be secreted out into the medium in yeast cells, making it easy to isolate and purify the protein. In particular, the present invention provides IL-1Ra with enhanced biological activity, which renders it applicable for therapeutic purposes.

The present invention provides a simple, cost effective procedure for producing IL-1Ra using Pichia pastoris (P. pastoris) as a expression host. As P. pastoris has a Generally Regarded As Safe (GRAS) status, the inventors have utilized it within the stated limits and have successfully expressed the protein to about 12-17% of the total protein. Moreover, the process provides a single step of purification followed by ultrafiltration to yield a pure protein with no native contaminant proteins.

In one embodiment, the present invention provides a method of producing a purified Interleukin-1 receptor antagonist (IL-1Ra) using a Pichia pastoris (P. pastoris) expression system, comprising the steps of: a) obtaining cDNA comprising a nucleic acid encoding an IL-1Ra protein, wherein the cDNA is prepared from RNA obtained from HS-5 cells; b) inserting the cDNA into a plasmid comprising a GAP promoter; c) transforming a P. pastoris host cell with the plasmid; d) expressing IL-1Ra protein in the host cell; and e) purifying the IL-1Ra protein; wherein the method involves continuous fermentation, and at least 10% of total protein secreted by the host cell into the fermentation medium is IL-1Ra protein.

In another embodiment, the nucleic acid encoding an IL-1Ra protein comprises a sequence chosen from GenBank Accession No. NM-173842, NM-173841, NM-000577, NM-173843 or EF140714.

In another embodiment, the nucleic acid encoding an IL-1Ra protein comprises a sequence of GenBank Accession No. NM-000577.

In another embodiment, the P. pastoris host cell is x-33.

In another embodiment, the plasmid is pGAPZαA.

In another embodiment, prior to step b) the method further comprises the steps of: a) ligating the cDNA to a first plasmid; b) inserting the first plasmid into a first host cell; c) recovering a replicated first plasmid harboring an IL-1Ra cDNA insert; d) excising the IL-1Ra cDNA insert from the first plasmid and inserting into a second plasmid; e) transforming a second host cell with the second plasmid; and f) recovering a replicated second plasmid expressing IL-1Ra.

In another embodiment, the first plasmid is pGEMT and the second plasmid is pET24a.

In another embodiment, the first and second host cells are selected from the group comprising XL-1 and TOP 10F′.

In another embodiment, prior to step b) the method further comprises amplifying the cDNA of step a) using PCR with primers selected from nucleic acid molecules comprising SEQ. ID NO.: 1, SEQ. ID. NO.: 2, SEQ. ID. NO.: 3, or SEQ. ID NO.: 4

In another embodiment, prior to step c), the plasmid is linearized with Avr II.

In another embodiment, at least 12-17% of total protein secreted by the host cell into the fermentation medium is IL-1Ra protein.

In another embodiment, the method further comprises a step of determining whether the purified IL-1Ra protein prevents IL-1β binding on MCF-7 cells.

In another embodiment, the method further comprises a step of determining whether the purifying IL-1Ra protein prevents MCF-7 cells from proliferating.

### BRIEF DESCRIPTION OF FIGURES

FIG. 1 illustrates the PCR amplification of cDNA for IL-1Ra from the HS-5 mammalian cell line. Lane #1: 100 bp ladder (NEB); Lane #2: no amplification seen with pfu enzyme in uninduced HS-5 cells; Lane #3: no amplification seen with pfu enzyme in induced HS-5 cells; Lane #4: amplification of IL-1Ra from uninduced HS-5 cells; Lane #5: no amplification seen with HS-5 cells induced with PMA; Lane #6: shows the negative control (no template).

FIG. 2 illustrates the expression profile of IL-1Ra/pET24a in BL21 (DE3) codon plus host strain; Lane #1: low molecular weight marker (Sigma); Lane #2: clone 1, before induction; Lane #3: clone 1, 2 hrs. after induction; Lane #4: clone 1, 4 hrs. after induction; Lane #5: clone 2, before induction; Lane #6: clone 2, 2 hrs. after induction; Lane #7: clone 2, 4 hrs. after induction; Lane #8: clone 3, before induction; Lane #9: clone 3, 2 hrs. after induction; Lane #10: clone 3, 4 hrs. after induction.

FIG. 3 illustrates the expression profile of IL-1Ra/pET24a in BL21(DE3) pLysS host strain; Lane #1: low molecular weight marker (Sigma); Lane #2: clone 1, before induction; Lane #3: clone 1, 3 hrs. after induction; Lane #4: clone 2, before induction; Lane #5: clone 2, 3 hrs. induction; Lane #6: clone 3, before induction; Lane #7: clone 3, 3 hrs. after induction; Lane #8: clone 4, before induction; Lane #9: clone 4, 3 hrs. after induction.

FIG. 4 illustrates the PCR amplification product of cloned IL-1Ra (NM-00057) into the pGEMT easy vector with ends compatible for pGAPZαA vector A. Lanes #1 and #2 represent the Taq PCR product, Lanes #3 and #4 represent the pfu PCR product, and Lane #5 represents the 1 Kb DNA ladder (NEB).

FIG. 5 illustrates the EcoR1 digestion of recombinant pGEMT clones T1-T12. Lane #1: 1 Kb DNA ladder (NEB); Lane #2: undigested IL-1Ra/pGEMT (clone T1); Lane #'s 3-14: EcoR1 digested IL-1Ra/pGEMT (clones T1 to T12); Lane #15: 100 bp DNA ladder (NEB).

FIG. 6 illustrates the EcoR1 digestion of the pGEMT easy vector and the pGEMT easy/IL-1Ra plasmid carrying the IL-1Ra insert. Lane #1: 1 kb DNA ladder (NEB); Lane #2: EcoR1 digested and gel eluted clone T2; Lane #3: Eco R1 digested and gel eluted clone T3; Lane #4: EcoR1 digested shrimp alkaline phosphatase (SAP) treated pGAPZαA; Lane #5: EcoR1 digested calf intestinal phosphatase (CIP) treated pGAPZαA; Lane #6: 100 bp DNA ladder.

FIG. 7 illustrates PCR amplification of IL-1Ra using pGAPZαA forward primer and the reverse primer for IL-1Ra; Lane #1 depicts the 1 Kb ladder (NEB), Lane #'s 3-14 depicts PCR products of positive clones cl. 3, 7, 8, 10, 16, 19, 20, 22, 25, 29, 30, 31, respectively, Lane #15 depicts the negative control (no template), and Lane 16 depicts the 100 bp ladder (NEB).

FIG. 8 illustrates the alignment view between the IL-1Ra gene cloned in pGAPZαA and the standard gene from NCBI for IL-1Ra (NM-00057), where TPG 677 F and TPG 677R-RC represent the two strands of the cloned plasmid DNA. The nucleotides underlined at the 5′ terminus shows the EcoR1 site followed by Glu Ala repeats, followed by the IL-1Ra start codon, CGA. The gene is shown ending with stop codon, TAG (underlined).

FIG. 9 illustrates the genomic DNA PCR of the recombinant P. pastoris x-33 strain showing integration of the IL-1Ra gene into its host genome. Lane #1: DNA ladder (1 kb); Lane #2: negative control; Lane #3: PCR product of clone 1 with gene specific primers; Lane #4: PCR product of clone 7 with gene specific primers; Lane #5: positive control. PCR of genomic DNA was performed to check for integration of IL-1Ra gene. The PCR products were run on a 1.2% agarose gel. As expected, the 500 bp amplification could be seen in clones 1 and 7.

FIG. 10 illustrates the SDS-PAGE profile of the partially purified IL-1Ra protein. Lane 1: Sea Blue Wide Range Marker (Invitrogen); Lane #2: recombinant IL-1Ra standard from E. coli (Propectany); Lane #3: purified protein from pGAPZαA Cl.7.

FIG. 11 illustrates the western blot analysis of proteins secreted from P. pastoris into media. Lane #1: Rainbow Marker (Invitrogen) RPN 755; Lane #2: vector control (pGAPZαA/x-33); Lane #3: 24 hour supernatant of clone 1; Lane #4: 48 hr supernatant of clone 1; Lane #5: 72 hr supernatant of clone 1; Lane #6: 24 hr supernatant of clone 7; Lane #7: 48 hr supernatant of clone 7; Lane #8: 72 hr supernatant of clone 7; Lane #9: IL-1Ra standard (Prospectany).

FIG. 12 illustrates the Q-Sepharose eluate after it has been silver stained. Lane #1: RPN-55 Rainbow marker (Invitrogen); Lane #2: purified IL-1Ra; Lane #3: Q-Sepharose load.

FIG. 13 illustrates the effect of IL-1beta on cell proliferation in media containing MCF-7 cells and the subsequent antagonist effect of IL-1Ra on the same. Relative cell number was measured by the BrDU assay. MCF-7 cells were cultured in serum free medium for 48 hours and then IL-1beta was added (50 ng/ml), which inhibited the cell growth. Upon adding IL-1Ra (test and standard), the cells showed proliferation (p<0.05).

### DETAILED DESCRIPTION OF THE INVENTION

The present invention provides an efficient process and methodology for expressing and producing Interleukin-1Ra (IL-1Ra) protein in an expression system. The present invention also provides methodology for purifying and characterizing the IL-1Ra protein. The present invention provides a simple and economical process for producing a IL-1Ra that is commercially viable, wherein the process can produce high levels of recombinant IL-1Ra protein for a variety of pharmaceutical and industrial purposes.

In one embodiment of the present invention, methodology is provided for producing IL-1Ra in high yields, comprising the steps of:

1. Constructing cDNA
2. Inserting the cDNA in a plasmid
3. Expressing a corresponding protein in yeast
4. Purifying the protein
5. Identifying and/or assaying the protein
6. Determining activity of the protein

In another embodiment, the present invention provides an efficient process for producing IL-1Ra that is stable, durable and cost-effective.

### Human Stromal-5 (HS-5) Cells

The HS-5 cell line is an adherent human stromal cell line isolated from bone marrow (ATCC). The cells can be maintained as a monolayer culture in DMEM medium.

### Interleukin-1Ra (IL-1Ra)

There are at least four isoforms of human IL-1Ra: NM-173842 (isoform 1), NM-173841 (isoform 2), NM-000577 (isoform 3), and NM-173843 (isoform 4). These 4 sequences are alternatively spliced transcript variants. Isoform 2 is the longest of the four. Isoform 3 lacks an in-frame coding segment as compared to isoform 2. Isoform 4 contains a distinct internal exon as compared to isoform 2. This results in translation from a downstream start codon. Isoform 4 also has shorter N-terminus as compared to isoform 2.

In one embodiment, NM-000577 (isoform 3) is used as the sequence for designing primers to amplify IL-1Ra cDNA from a cell line. One such cell line is HS-5.

### Expression Systems

In one embodiment, IL-1Ra DNA is amplified from a cell line and cloned into an expression vector.

In another embodiment, the cell line is the HS-5 cell line. Example I is a non-illustration limiting of this process with the NM-000577 variant.

In another embodiment, the expression vector is the pGEMT Easy expression vector. Example II is a non-limiting illustration of this process. Clones produced from this process can be sequenced to check for absence of PCR errors, and the sequence can be matched with human IL-1Ra sequences from NCBI using Omiga software for alignment. All final constructs can be sequenced to verify that no misincorporation occurs during PCR amplification.

In another embodiment, a transformant is prepared by introducing the expression vector into a suitable or appropriate host by the conventional method of Hanahan (Hanahan, J. of Mol. Biol. (1993) Vol. 166, pp. 557-580).

In another embodiment, a transformant is prepared by transfecting the expression vector into a host cell using recombinant DNA technology.

In another embodiment, glyceraldehydes-3 phosphate dehydrogenase enzyme (GAP) promoter is used in expression vectors. In a GAP promoter expression system, cloned heterologous protein is expressed during cell growth if the protein is not toxic for the cell. This system requires no washing to remove non-methanolic carbon sources, and no accurate optimization of the culture conditions as in methanol induction phase. This system is more suitable for large-scale production because the hazard and cost associated with the storage and delivery of large volumes of methanol in other systems are eliminated. Expression vectors having GAP promoters in P. pastoris allow for continuous production of recombinant product, avoiding the traditional P. pastoris fed-batch fermentations using a methanol inducible system. The features of the GAP expression system may contribute significantly to the development of cost-effective methods for large-scale production of heterologous recombinant proteins via fermentation of genetically engineered P. pastoris, where proteins are folded correctly and secreted into the medium. In fact, the inventors found that the yield of the desired protein obtained with this GAP promoter was found to be greater than that obtained with the AOX promoter.

In another embodiment, the IL-1Ra DNA is linearized with Avr II. Linear DNA can generate stable transformants via homologous recombination between the transforming DNA and region of homology within the cell genome.

In another embodiment, the host cell transformed with the expression vector is cultured under conditions that promote expression.

In another embodiment, the expression is promoted extracellularly.

In another embodiment, the host cell is bacteria.

In another embodiment, the bacteria is Escherichia coli (E. coli).

In another embodiment, the host cell is yeast.

In another embodiment, the yeast is Pichia pastoris (P. pastoris), also known as Komagataella pastoris. P. pastoris can use methanol as a carbon source in the absence of glucose. Receptor binding activity of our purified protein indicated that it was correctly folded and functional.

The successful hyper-expression of glycoprotein hormones using the P. pastoris expression system has many important implications. Shake-flask conditions are sub-optimal for protein secretion because of lack of pH control, inadequate aeration of cultures and inability of control of feeding of carbon sources at optimal rates, etc. The cell density obtained is much lower, when compared to a fermentor. Thus, by maintaining the per cell productivity constant in fermentation, it should be possible to achieve a 20-30 fold increase in the yield of the protein of interest. When scaling up for industrial levels, this process is safer and devoid of the methanol hazards associated with the inducible vector systems like pPIC 3.5 or pPIC 9k.

In another embodiment, IL-1Ra is subcloned into a pET24a expression vector at Nde-1 and BamH-1 sites.

In another embodiment, the bacteria is E. coli BL21(DE3). Example III is a non-limiting illustration of this process to check the protein's expression in E. coli. In another embodiment, the bacteria is E. coli BL21(DE3) codon plus is transformed with the expression vector. Recombinant IL-1Ra can be produced by culturing the transformed recombinant E. coli under suitable conditions.

In another embodiment, the bacteria is E. coli BL21(DE3) pLysS. In non-limiting Example III, the inventors discovered that E. coli BL21 (DE3) and E. coli BL21 (DE3) codon plus provide very good expression of IL-1Ra, but all samples showed leaky expression, as exemplified by SDS-PAGE and western blot analysis (data not shown). To control leaky expression, the inventors transformed the BL21 (DE3) pLysS E. coli strain. As performed by the inventors, induction by IPTG and subsequent analysis by SDS-PAGE showed very good expression of the protein with no leaky expression. Western blot analysis also confirmed the presence of IL-1Ra. The protein was sent for N-sequencing terminal sequencing (Prosite), with results showing that the amino sequence read in accordance with the DNA sequence (NM-00057).

In another embodiment, the bacteria is E. coli TOP 10F′.

In another embodiment, IL-1Ra is subcloned into pGAPZαA.

In another embodiment, the host cell is P. pastoris X-33.

In another embodiment, DNA isolated from the E. coli TOP 10′F. cell is transformed into a P. pastoris X-33 cell. Example IV is a non-limiting illustration of this process, whereby positive clones, determined by sequence analysis, are taken forward for transformation into P. pastoris. As performed by the inventors, Avr II linearized pGAPZαA/IL-1Ra was transformed into log phase X-33 cells (0.8 OD) and the resulting transformants were characterized for presence of gene integration. Twelve Zeocin resistant transformants were checked for the presence of IL-1Ra using PCR. Clones were selected based on their high resistance to Zeocin, since high resistance means high copy number (Pichia EasyComp Kit, Invitrogen).

These clones were simultaneously plated on YEPDS plates containing 500, 1000 and 2000 μg/ml Zeocin. Twelve colonies grew on Zeocin plates containing 500 μg/ml of the drug. Two colonies, clone 1 (Cl.1) and clone 2 (Cl.7), grew on 1000 μg/ml Zeocin. Cl.7 also grew on 2000 μg/ml Zeocin. In fact, Cl.7 grew on 500, 1000 and 2000 μg/ml Zeocin plates, leading the inventors to use Cl.7 in expression studies. Genomic PCR showed the presence of a 500 bp band corresponding to the IL-1Ra gene in both Cl.1 and Cl.7. Proteins, when separated by SDS/PAGE (17%), did not show any bands by coomassie staining. However, immunoreactive protein was detected by polyclonal antibody against IL-1Ra raised in goat (R&D Systems).

Alkaline phosphatase (ALP) conjugated antibody was used as the secondary antibody and detected by substrate BCIP-NBT addition. Levels of IL-1Ra in the P pastoris culture supernatant was estimated from western blot as being in the range of 50-60 μg/ml. The total protein content of the P. pastoris supernatant was estimated by the Bradford method to be 300 μg/ml, suggesting that 12-17% of the secreted protein was the protein of interest. The protein was partially purified for the purpose of N-terminal sequencing, which showed two bands on SDS-PAGE when stained with coomassie blue. To clear any doubt of glycosylation, the inventors performed a N-glycosidase treatment of the protein and found that it migrated at the same level as that of the untreated sample (data not shown). This lead the inventors to believe that the other band seen could be another similar protein with a slightly higher molecular weight. The results show that IL-1Ra is a protein with a molecular weight of about 20 kD and can be successfully expressed by x-33 cells as a secretive protein. One-step purification followed by ultrafiltration yielded quite a pure protein with very few contaminant proteins.

Fermentation of P. pastoris

In another embodiment, the present invention provides a fermentation process that produces a relatively low cell density. The present invention provides a 20-30 fold increase in the yield of the protein of interest by maintaining the per cell productivity constant.

The present invention provides a total concentration of the desired protein in culture supernatant from transfected cells in the range of 50-60 μg/mL.

### Purification of IL-1Ra

In one embodiment, the present invention provides methodology for purifying IL-1Ra protein from culture media.

In another embodiment, the IL-1Ra protein can be secreted directly into medium by P. pastoris, making it easy to isolate and purify the protein. In the non-limiting examples that follow, as P. pastoris has a Generally Regarded As Safe (GRAS) status, the inventors have utilized it within the stated limits and has successfully expressed the protein to about 12-17% of the total protein. In a related embodiment, the process provided by the present invention allows a single step of purification followed by ultrafiltration to yield a pure protein with no native contaminant proteins.

In another embodiment, cell extracts are obtained by exposing them to lysozyme treatment, digestion, freezing and thawing, ultrasonication or French press process. This can followed by solubilization of the extracts, ultrafiltration, dialysis, ion exchange chromatography, gel filtration, electrophoresis or affinity chromatography.

As used herein the term “IL-1Ra” means IL-1Ra polypetides and analogs thereof having substantial amino acid sequence identity to the different isoforms of IL-1Ra. As used herein “expression vectors” capable of expressing proteins composed of IL-1Ra are inserted into vectors containing appropriate promoters. Such promoters include Lac, trp, tac, P1, T3, T7, SP6, SV40, and GAP. DNA is ligated to these promoters and ribosomal binding sites, followed by the construction of vector system by inserting them into various plasmids. DNA encoding the desired proteins or peptides are inserted into the 3′ terminus of these vectors and the resulting expression vectors are introduced into appropriate hosts. Constructed transformants are cultured to produce the desired fusion proteins.

### Bioactivity Assays

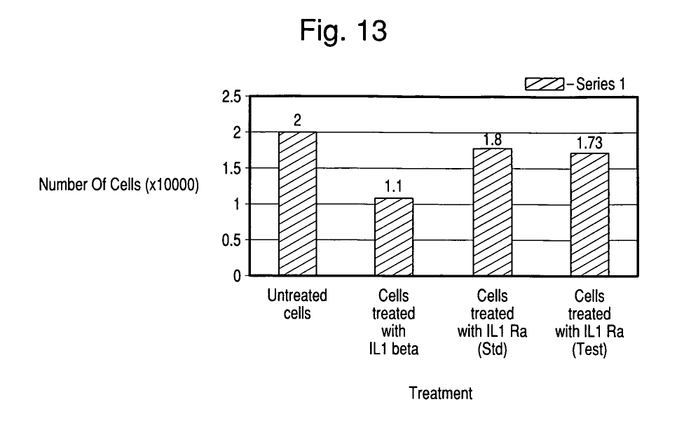

In one embodiment of the present invention, bioactivity studies can be performed on the purified proteins. In non-limiting Example V, the inventors determined that the partially purified protein was active. See FIG. 13 providing an illustration of these studies. Experiments in the lab proved that 50 ng of IL-1beta could inhibit the proliferation of MCF-7 cells. An amount of 50 ng of IL-1beta and increasing concentrations of IL-1Ra (test protein) were added to 20×10³ cells of MCF-7. It was found that 40 ng of test protein showed activity. Similarly, in another set of wells, 50 ng of IL-1 beta and increasing concentrations of IL-1Ra (standard from Prospectany) was added to 20×10³ cells of MCF-7, to determine which concentration optimally helped in proliferation. It was found that 20 ng of standard IL-1Ra could competitively inhibit IL-1beta from blocking the receptors. Both test and standard IL-1Ra blocked the IL-1 receptors, denying access to IL-1 beta resulting in the proliferation of the cells.

The following examples are included to demonstrate select embodiments of the invention. It should be appreciated by those of skill in the art that the techniques disclosed in the examples represent techniques discovered by the inventor to function well in the practice of the invention, and thus can be considered to constitute best modes for its practice. However, those of skill in the art should, in light of the present disclosure, appreciate that many changes can be made in the specific embodiments and still obtain a like or similar results without departing from the scope of the invention.

### EXAMPLE I

### Construction and Amplification of IL-1Ra cDNA

1. Total RNA Preparation of IL-1Ra from HS-5 Cell Line

Total RNA for IL-1Ra was obtained from Human Stromal-5 cells (HS-5 cells). The HS-5 cells were lysed and the total lysate was applied to an RNEasy mini column to obtain elutants comprising the IL-1Ra RNA.

2. cDNA Preparation of IL-1Ra from Total RNA

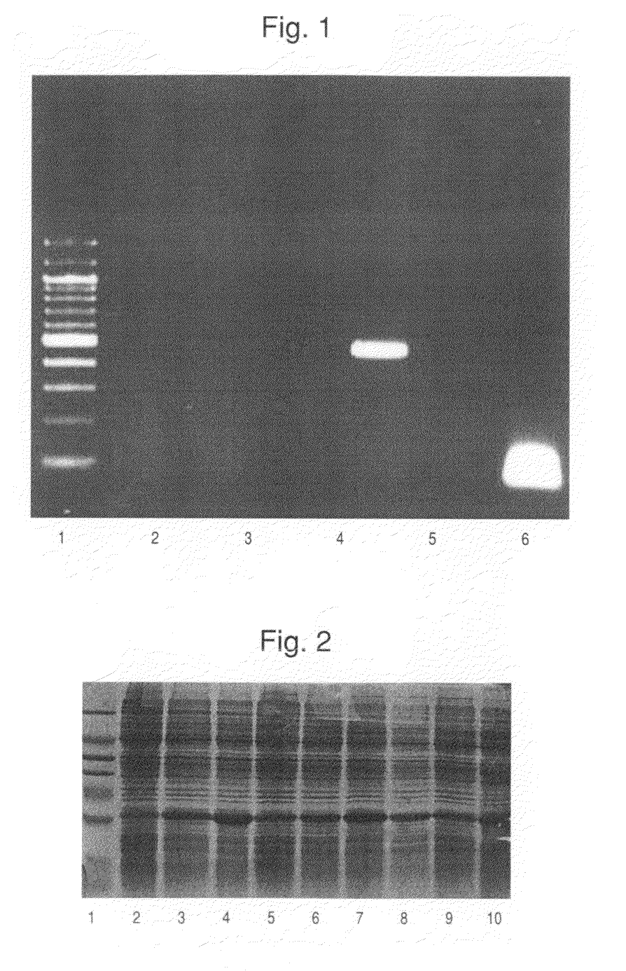

Total RNA was isolated from the HS-5 cell line using the RNeasy Mini kit (Qiagen). The RNA obtained from the RNeasy mini column was used to prepare cDNA using the Omniscript RT kit (Qiagen). The process for preparing the cDNA required performing PCR of the eluted RNA using dNTP mix and Oligo dT primers. The cDNA obtained was then used as a template further for PCR amplification. See FIG. 1.

3. PCR Amplification of IL-1Ra cDNA with Nde-1 and Bam H-1

PCR amplification was carried out using the above cDNA template and following primers:

Forward primer for cloning IL-1-Ra (ANAKINRA) with
Nde-1 at the 5′ end:

(SEQ. ID NO.: 1)

TPG 317-5′d (CATATGCGACCCTCTGGGAGAAAATC)3′
Reverse primer for cloning IL-1-Ra (ANAKINRA) with
Bam H1 site at 3′ end:

(SEQ. ID NO.: 2)

TPG-318-5′d (GGATCCTCTACTCGTCCTCCTGGAAG)3′

The touchdown PCR condition was 14 cycles of 30 sec. at 95° C., 30 sec. at 69.7° C. (with a decrease of 0.5 degrees per cycle), 50 sec. at 72° C., followed by 19 cycles of 30 sec. at 95° C., 30 sec. at 62.7° C., and 50 sec. at 72° C., after denaturing for 5 min. at 94° C. The amplified 459 bp band was purified with a Qiaquick Gel Extract Kit (Qiagen) and subcloned into pGEMT vector (Promega). The clone was sequenced to check for absence of PCR errors and the sequence matched with human IL-1Ra sequence from NCBL (NM-00057), using Omiga software (Accelrys).

### EXAMPLE II

### Preparation of Plasmids

1. Elution of the Band from Gel

The band of DNA corresponding to IL-1 Ra was eluted out of the gel using manufacturer's instructions of the Qiagen gel extraction kit.

2. Ligation of the DNA Fragment with pGEMT Vector

The eluted DNA fragment was ligated to a pGEMT vector in the presence of T4 DNA ligase, rapid ligation buffer, at about 4° C., for about 16-18 hours. The result was a IL-1Ra/pGEMT plasmid ligation mixture.

3. Transformation of IL-1Ra/pGEMT into XL-1 Competent Cells

The IL-1Ra/pGEMT plasmid ligation mixture was transformed into competent bacterial XL-1 cells following standard transformation methods. The transformants were plated onto LB-IPTG-AMP plates and the plates were incubated at 37° C. overnight.

### 4. Screening of the Colonies

Plenty of white colonies were seen on the plate, out of which 10 were inoculated into LB broth for plasmid preparation. Plasmids were made from these cultures by the QIAprep Spin Miniprep Kit (Qiagen).

### 5. Restriction Mapping of the Positive Clones

The plasmids were digested with EcoR1. All of the plasmids released 500 bp fragments.

### 6. Sequencing Positive Clones

Positive clones which released 500 bp fragment were sent for sequencing. Sequences were perfectly aligned with the IL-1Ra sequence of Genbank (NM-00057).

### EXAMPLE III

### Expressing IL-1Ra in E. Coli

1. Sub-cloning of IL-1Ra into a pET24a Vector

The positive clones from Example II were subjected to Nde-1/BamH1 digestion and resolved on a 1.5% agarose gel. The Nde-1/BamH1 fragment was eluted from the gel and ligated to the expression vector, pET24a. A mixture of 5 μl of the insert, 1 μl of the vector, 1 μl of the ligase buffer and 1 μl of the T4 DNA ligase were provided in a tube and incubated at 16° C. overnight.

2. Transformation of IL-1Ra/pET24a into TOP10F′ Cells

The IL-1Ra/pET24a plasmid ligation mix was transformed into competent bacterial TOP10F′ cells following standard transformation protocols. The transformants were plated onto LB-kananycin plates. The plates were incubated at 37° C. Eight colonies were chosen for plasmid preparation and further analysis. The colonies were inoculated into LB broth and plasmids were made by the Qiagen method, disclosed above. The plasmids were digested with Nde-1/BamH-1, six of which released the IL-1Ra fragment of the expected size. The DNA from the six plasmids were sent for sequencing and returned with perfect matches to the known NM-00057 IL-1Ra sequence.

### 3. Screening of the Colonies

Numerous colonies were seen on the plate and 20 clones, clones 1- to 20 (Cl.1 to Cl.20) were inoculated into LB broth for plasmid preparation. Plasmids were made from these cultures by the Qiagen method, as per the manufacturer's instructions.

### 4. Restriction Mapping of the Positive Clones

The clones were subjected to Nde-1-BamH-1 digestion and the clones that released the 500 bp IL-1Ra fragment (corresponding to NM-00057) were isolated.

5. Transformation of Positive Plasmids into Expression Host BL21(DE3)

A volume of 3 μl of the plasmid preparation was transformed into competent BL21 (DE3) cells by standard transformation protocols. The transformants were plated on LB+kanamycin plates. Numerous colonies could be seen on the plate. A couple of the positive clones (named as clone 1 and 2) were taken for expression studies.

### 6. Expression

BL21 (DE3) transformed with pET24a vector alone served as the vector control. Three 500 ml flasks containing 100 ml of LB kanamycin were inoculated with overnight cultures of clone 1, clone 2 and the pET24a transformed into BL21(DE3) as vector control. The initial O.D. was adjusted to 0.1 by taking appropriate volume of inoculum. The culture was incubated at 37° C. with shaking until the O.D. reached 0.8. The cultures were then induced with 0.5 Mm IPTG final concentration and incubated at 37° C. with constant shaking. Samples were collected at 0 hrs, 3 hrs and 5 hrs, post induction. Culture volumes corresponding to 1 O.D. were taken and spun down. The pellets were stored at −20° C. (the cultures after 5 hrs of induction were spun down at 4° C. and the pellets stored at −70° C.).

The collected pellets were lysed by adding 100 μl of B-PER reagent following the instructions of the B-PER Bacterial Protein Extraction Reagent (Pierce). A volume of 20 μl of 6×SDS loading dye was added and samples were boiled for 10 minutes. A volume of 10 ul of the sample was then loaded for SDS-PAGE and 5 ul of the same sample was loaded for Western blot, and both samples were run on 15% gels.

The appearance of a highly expressed protein only in the IL-1Ra clones (1 and 2) and not in the control vector confirmed that IL-1Ra expression levels were very high even in the shake flask levels.

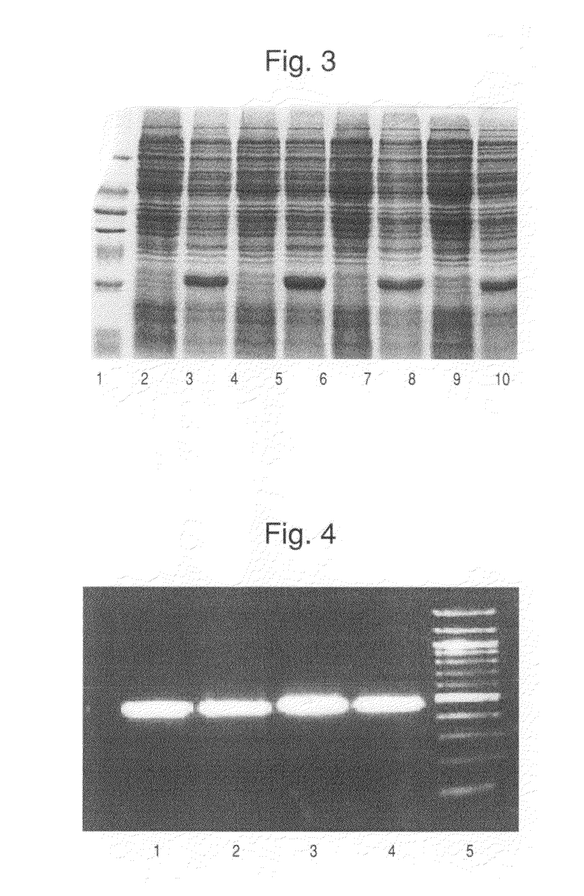

Although the expression of the heterologous protein IL-1Ra was very high in both BL21(DE3) and BL21(DE3) codon plus hosts (FIG. 2), expression was leaky, i.e., there was expression seen even in the uninduced lanes. Hence, the cloned IL-1Ra gene was transformed into BL21(DE3) pLysS strain (Stratagene). See FIG. 3, illustrating four transformed clones that were selected for further studies.

To these four clones, a volume of 100 μl of B-PER lysis buffer was added to each sample and incubated on ice for 10 minutes. A volume of 20 ul of 6×SDS PAGE dye was added to all the tubes and boiled for 10 minutes. 15 μl and 7 μl of the above samples were loaded onto 15% SDS-PAGE gel for coomassie blue staining and western blot respectively. For western blotting, the proteins were transferred onto nitrocellulose membrane at 100 V for 1 hour and then blocked by 10% skimmed milk powder in TBST for 1 hour. Probing was performed with monoclonal primary antibody and secondary antibody raised in mouse and conjugated with ALP.

As expected, there was no detectable expression of IL-1Ra in the uninduced lanes in both the coomassie stained and Western blot gels. See FIG. 3. The level of expression was also comparable to BL21(DE3) and BL21(DE3) codon plus.

### EXAMPLE IV

### IL-1Ra Expression in P. Pastoris

1. PCR Amplification of IL-1Ra cDNA

PCR amplification was performed using IL-1Ra/pET24a from clone 20 (Cl.20) from Example III, with primers TPG 432 and 433. The Glu-Ala repeats were reconstructed beyond the EcoR1 site in the primer for proper cleavage of Saccharomyes cerevisiae alpha mating signal sequence:

(SEQ. ID. NO.: 3)

TPG-432-5′d (GAATTCGAGGCTGAAGCTCGACCCTCTGGGAGAAA
ATC)3′

(SEQ. ID. NO.: 4)

TPG-433-5′d (CTCGAGCTACTCGTCCTCCTGGAAGT)3′

The reaction mix included 1 μL of template, 2.5 μL of each primer, 19 μL of water and 25 μL of Abgene's master mix. The PCR conditions included the following sequence for one cycle: 95° C. for 2 minutes, 95° C. for 30 seconds, 62° C. for 30 seconds, 72° C. for 50 seconds, and followed by extension at 72° C. for 5 minutes. This was cycled for 25 times. See FIG. 4. The band was extracted and cleaned using the Qiagen gel extraction kit.

2. Ligation of the Eluted DNA into pGEMT and pGAPZαA Vectors

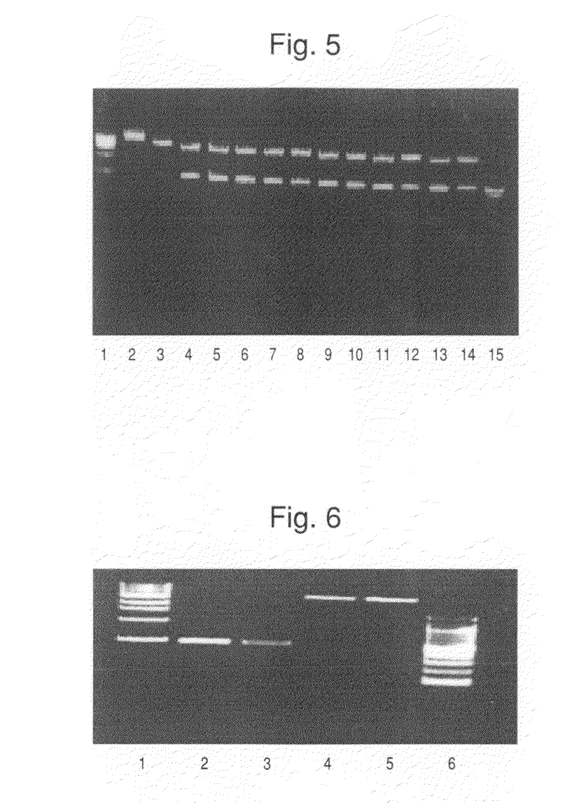

The cleaned IL-1Ra fragment discussed in the previous paragraph was ligated to a pGEMT easy vector to produce a ligation mix and incubated at 4° C. The ligation mix was transformed into E. coli TOP 10F′ cells and the transformants were plated on LB+IX+amp plates. Numerous colonies were observed on the plate. Plasmids were made from fourteen of them and digested with EcoR1 to check for the insert. See FIG. 5.

Large scale digestion was performed on two positive clones, T2 and T3. Digestion was also performed on the vector pGAPZαA, which is a yeast expression vector containing a relevant EcoR1 restriction site. The EcoR1 digested and cleaned vector was treated with calf intestinal phosphatase (CIP), followed by a cleaning using the MinElute Reaction clean up kit (Qiagen). See FIG. 6, showing both shrimp alkaline phosphatase (SAP) and CIP treated vectors.

The EcoR1 Fragment of clone T2 was ligated to a pGAPZαA vector using T4 DNA ligase and incubated overnight at 16° C. The ligation mix was transformed into TOP10F′ cells and plated on LB low salt+zeocin plates. Roughly 30 colonies could be seen on the plates. The vector control plate had no colonies. A colony PCR was performed for all the transformants, in order to check for the presence of insert as well as orientation. The pGAPZαA forward primer and reverse primer for IL-1Ra (NM-00057) were used. The PCR conditions included the following sequence for one cycle: 95° C. for 2 minutes, 95° C. for 30 seconds, 62° C. for 30 seconds, 72° C. for 50 seconds, and followed by extension for 5 minutes and cycled for 25 times. Twelve clones were positive for the insert in the right orientation.

A PCR was performed again to reconfirm the presence of insert using the plasmid as the template. All of the samples showed the expected band. Bands from Cl.3, Cl.20 and Cl.22 samples were sent for sequencing. The appropriate sequences used were:

(SEQ. ID. NO.: 3)

TPG-432-5′d (GAATTCGAGGCTGAAGCTCGACCCTCTGGGAGAAA
ATC)3′

(SEQ. ID. NO.: 2)

TPG-318-5′d (GGATCCTCTACTCGTCCTCCTGGAAG)3′

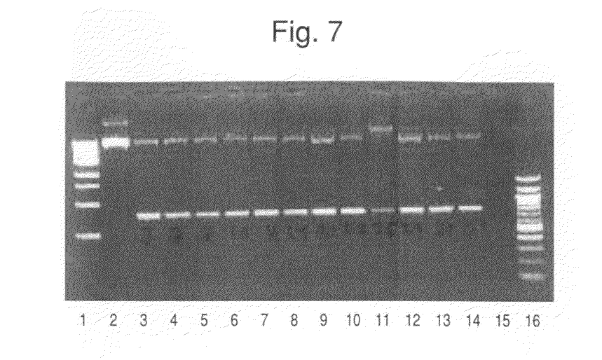

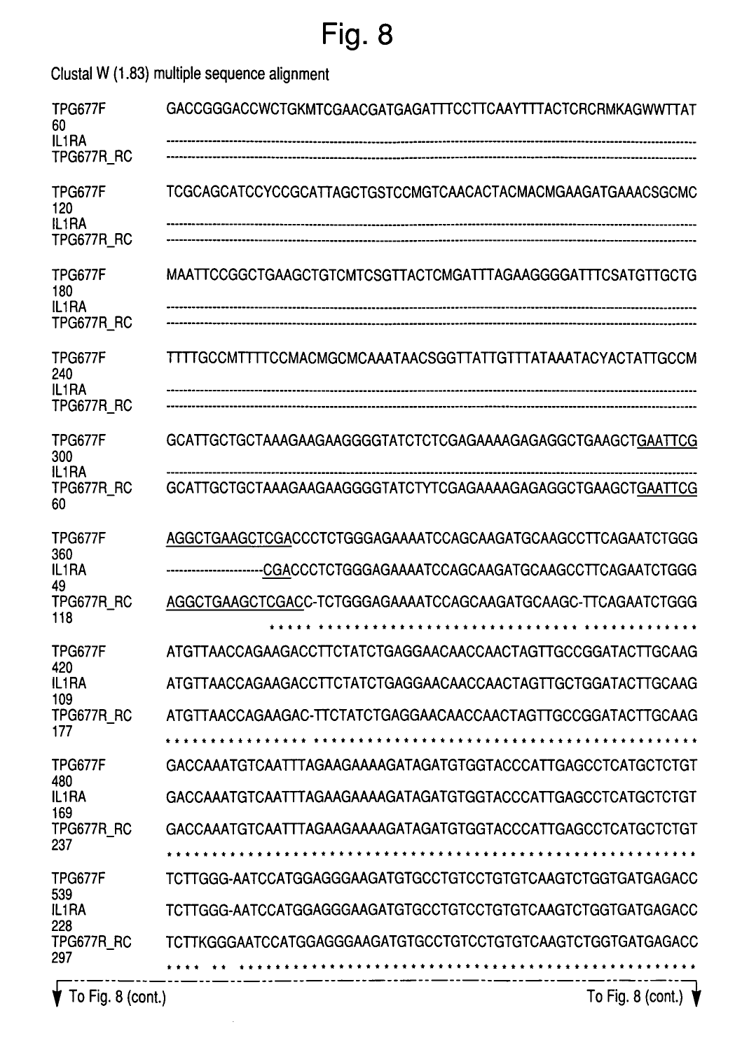

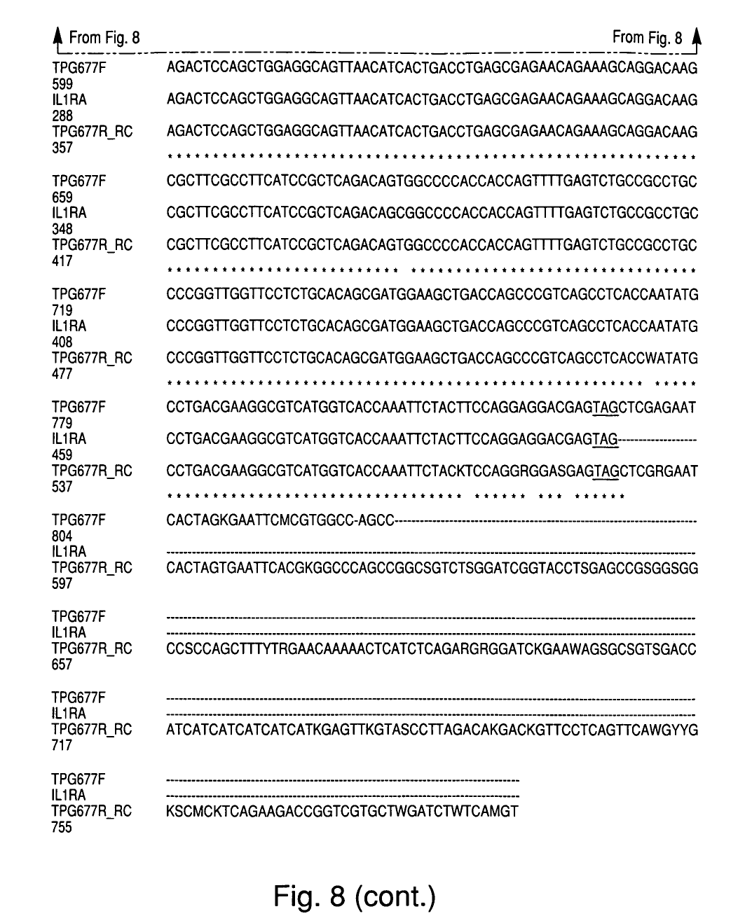

See FIG. 7. The sequence for Cl.20 was aligned correctly and was used for transformation into P. pastoris. See FIG. 7, lane 9 showing Cl.20 and FIG. 8 for the alignment of the cloned RL-1Ra with the standard IL-1Ra (NM-00057).

### 3. Screening of the Plasmids

Cl.20 was selected for further characterization. The DNA from the clone was linearized with Avr II. Avr II cuts in the GAP promoter region once to linearize the vector. Avr II was found not to cut in the IL-1Ra sequence. The linearization was checked on the gel and once completely linearized, was heat activated. After passing the DNA through MinElute reaction clean up kit (Qiagen), the DNA was eluted in sterile water.

4. Transformation of the Linearised IL-1Ra into Competent P. pastoris x-33 Cells

10 μL (˜8 μg DNA) of the linearlized IL-1Ra/pGAPZαA plasmid was mixed with 50 μl of x-33 cells, to which a solution of the Easy Comp Transformation kit (Invitrogen) was added and vortexed. The mixture was incubated at 30° C. for 1 hour with intermittent mixing every 15 minutes. The cells were heat shocked for 10 minutes and then split into aliquots of 525 μL each. The aliquots were mixed with 1 ml of YEPD liquid medium. After incubation, the cells were pelleted at 3000×g for 5 minutes at 37° C. for 2-4 days and spread on YEPD+zeocin plates and incubated.

### 5. Screening of the Colonies

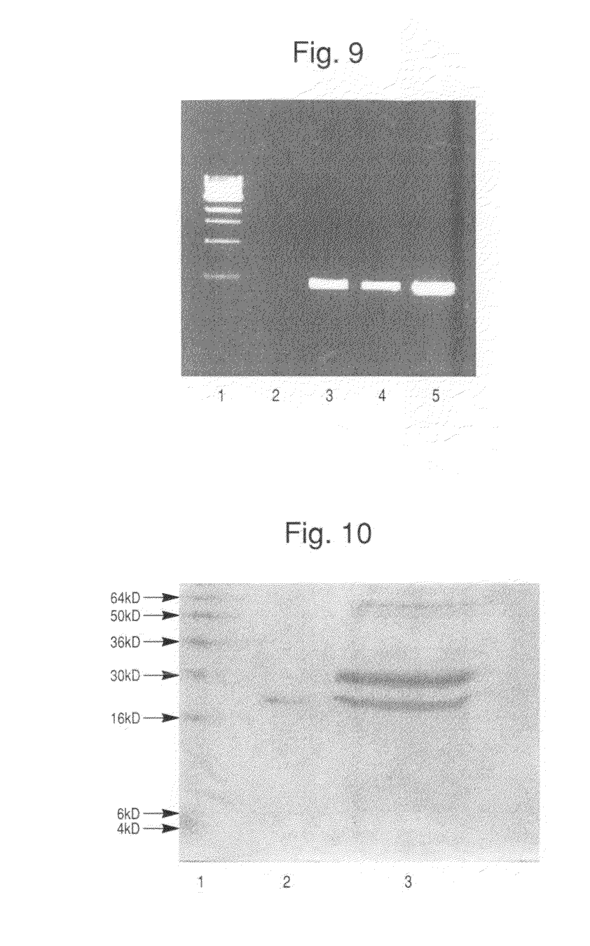

Zeocin resistant transformants were checked for the presence of IL-1Ra using PCR. See FIG. 9, illustrating the integration of IL-1Ra from C.1 (lane 3) and C.7 (lane 4) with the P. pastoris genome. The colonies growing on Zeocin were isolated for further studies. These clones were simultaneously plated on YEPDS plates containing 500, 1000 and 2000 μg/ml zeocin. Colonies growing on 1000 μg/ml Zeocin were isolated for further studies. Clone 7 (Cl.7) was seen to grow on 500, 1000 and 2000 μg/ml Zeocin plates.

### 6. Expression

Cl.7 was inoculated in 10 ml YPD and incubated at 30° C. with shaking overnight. A volume of 0.1 ml of the overnight culture was inoculated into 50 ml of YPD in a 250 ml flask and grown at 30° C. in a shaking incubator at 295 rpm. At 0 hr, 24 and 48 hours, 1 ml of the culture was taken in a 1.5 ml eppendorf tube. The cells were centrifuged at maximum speed for 3 minutes at 30° C. The supernatant was aspirated and assayed for expression of IL-1Ra.

### 7. SDS/PAGE Analysis and Western Blot Analysis

For coomassie blue staining, proteins were separated by SDS/PAGE (15%). Cell free supernatant (15 ul) from 1 ml culture was loaded in each well. Coomassie staining revealed IL-1Ra protein. See FIG. 10, illustrating the presence of IL-1Ra protein from Cl.7 as compared with standard recombinant II-1 Ra (Prospectany).

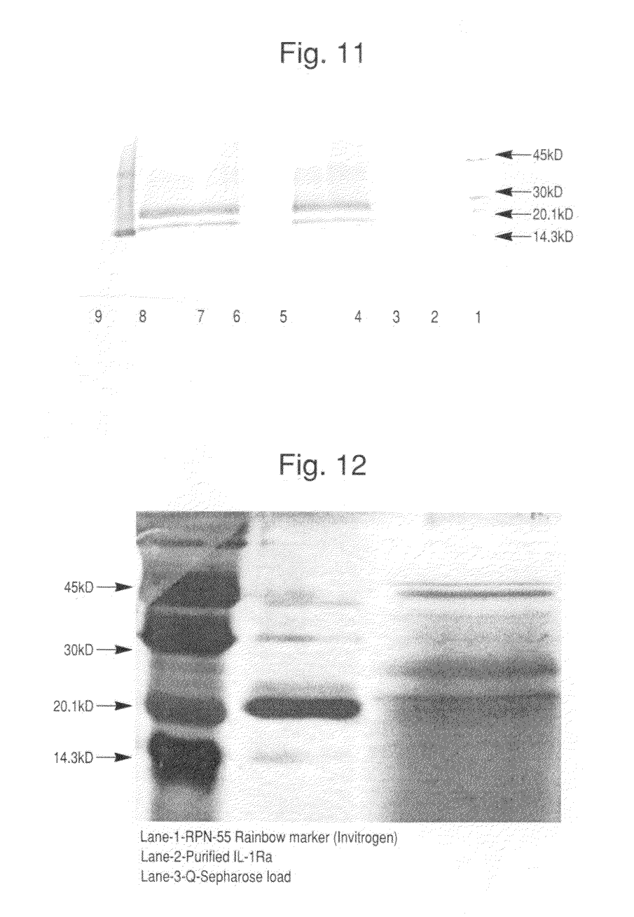

For western blotting, cellular and secreted proteins were separated by SDS/PAGE. The samples were boiled for 5 minutes in SDS-PAGE loading buffer. The proteins were separated by SDS/PAGE and transferred to a nitrocellulose membrane. Additional protein binding was blocked by incubation with 5% (w/v) skim milk for 18 hrs at 4° C. Immunoreactive protein was detected by monoclonal antibody against IL-1Ra. ALP-conjugated antibody was used as the secondary antibody and detected by substrate BCIP-NBT addition, as illustrated by FIG. 11.

### 8. Purification

After 48 hours of culturing, the IL-1Ra/pGAPZA from Cl.7 was spun down and the supernatant obtained was loaded onto a Q-sepharose column, which was washed with water and then equilibrated with 20 mM Tris buffer (pH 8.8). The column was washed with 20 mM Tris (pH 8.8) and the protein was eluted with 200 mM NaCl containing 20 mM Tris. This was followed by two elutions with 500 mM NaCl and 1 M NaCl, respectively. The eluted fractions were resolved on a 15% SDS GEL and silver stained. Only the 500 mM NaCl fraction gave good intensity of the IL-1Ra band. This fraction was loaded on an SDS-PAGE gel and stained with commassie. See FIG. 12. Protein concentrations were determined with the protein assay reagent (Biorad) by using bovine serum albumin as the standard. The purified protein from the isolated fraction showed a concentration of 45 mg/ml.

High expression levels have been obtained in continuous production of IL-1Ra by P. pastoris with a constitutive promoter (GAP promoter) in a 1.5 litre working volume fermenter using either glucose or glycerol as the carbon source. The fermentation could be extended for long periods of time, where excellent steady-state protein concentration and cell densities were achieved. No proteolytic degradation of the enzyme was seen in the continuous fermentation mode.

### EXAMPLE V

### Analysis of the Recombinant Protein

### 1. Qualitative N-Terminal Sequencing of the Protein

The partially purified proteins of the previous Examples III and IV were blotted onto a PVDF membrane at 90 V for 1 hour and then stained in 0.02% coomassie blue with 40% methanol and 5% acetic acid for 20 seconds. The membrane was then destained for up to 1 minute in 40% methanol and 5% acetic acid. The membrane was rinsed in water three times and dried between whatman papers. The band was then cut out and sent for sequencing. The N-terminal sequencing results indicated that yeast processed the signal sequence properly and released the IL-1Ra of correct N-terminal sequence.

### 2. Bioassay of IL-1Ra

a) Inhibitory Activity of IL-1beta on MCF-7 Cells

Cells were cultured in a total volume of 200 μl of 1% serum containing media. Recombinant human IL-1beta was added to a final concentration of 50 ng/ml. Both treatment and control tests were performed in three replicate wells. The relative number of cells was determined at 72 hours of incubation by incubating the cells with BrdU, for 12 hours before reading the plate. The optical density was measured at 430 nm. The absorption values were converted to cell numbers, which actually translates to the cell number. The amount of IL-1beta, which could inhibit the growth of MCF-7 cells, was determined from this experiment. It was found that an amount of 50 ng of IL-1 beta could inhibit the proliferation of MCF-7 cells.

b) Bioactivity of IL-1Ra

An amount of 50 ng of IL-1beta and increasing concentrations of IL-1Ra (Test) were added to MCF-7 cells (at an amount of 20×10³ cells) in a microtiter plate. Similarly, in another set of wells, 50 ng of IL-1beta and increasing concentrations of IL-1Ra (standard from Prospectany) was added to 20×10³ cells of MCF-7, to determine which concentration optimally helped in proliferation, and in turn indicating if the IL-1Ra protein was active or not. All the experiments were performed in triplicate. See FIG. 13, illustrating that MCF-7 cells treated with IL-1Ra subsequent to treatment with IL-1beta proliferated much more than cells treated with IL-1 beta only.

All of the compositions and methods disclosed and claimed herein can be made and executed without undue experimentation in light of the present disclosure. While the compositions and methods of this invention have been described in terms of certain embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention.

What is claimed is:

1. A method of producing a purified Interleukin-1 receptor antagonist (IL-1Ra) using a Pichia pastoris (P. pastoris) expression system, comprising the steps of:
a) obtaining cDNA comprising a nucleic acid encoding an IL-1Ra protein, wherein the cDNA is prepared from RNA obtained from HS-5 cells;
b) inserting the cDNA into a plasmid comprising a GAP promoter;
c) transforming a P. pastoris host cell with the plasmid;
d) expressing IL-1Ra protein in the host cell; and
e) purifying the IL-1Ra protein;
wherein the method involves continuous fermentation, and at least 10% of total protein secreted by the host cell into the fermentation medium is IL-1Ra protein.

2. The method of claim 1, wherein the nucleic acid encoding an IL-1Ra protein comprises a sequence chosen from GenBank Accession No. NM-173842, NM-173841, NM-000577, NM-173843 or EF140714.

3. The method of claim 1, wherein the nucleic acid encoding an IL-1Ra protein comprises a sequence of GenBank Accession No. NM-000577.

4. The method of claim 1, wherein the P. pastoris host cell is x-33.

5. The method of claim 1, wherein the plasmid is pGAPZαA.

6. The method of claim 1, wherein prior to step b) the method further comprises the steps of:
a) ligating the cDNA to a first plasmid;
b) inserting the first plasmid into a first host cell;
c) recovering a replicated first plasmid harboring a IL-1Ra cDNA insert;
d) excising the IL1-Ra cDNa insert from the first plasmid and inserting into a second plasmid;
e) transforming a second host cell with the second plasmid; and
f) recovering a replicated second plasmid expressing IL-1Ra.

7. The method of claim 6, wherein the first plasmid is pGEMT and the second plasmid is pET24a.

8. The method of claim 6, wherein the first and second host cells are selected from the group comprising XL-1 and TOP10F′.

9. The method of claim 1, wherein prior to step b) the method further comprises amplifying the cDNA of step a) using PCR with primers selected from nucleic acid molecules comprising SEQ. ID. NO.: 1, SEQ. ID. NO.: 2, SEQ. ID NO.: 3 or SEQ. ID NO.: 4.

10. The method of claim 1, wherein, prior to step c), the plasmid is linearized with Avr II.

11. The method of claim 1, wherein at least 12-17% of total protein secreted by the host cell into the fermentation medium is IL-1Ra protein.

12. The method of claim 1, wherein the method further comprises a step of determining whether the purified IL-1Ra protein prevents IL-10 binding on MCF-7 cells.

13. The method of claim 1, wherein the method further comprises a step of determining whether the purifying IL-1Ra protein prevents MCF-7 cells from proliferating.
